(19) United States
(12) Patent Application Publication
(10) Pub. No.: US 2024/0000745 A1
(43) Pub. Date: Jan. 4, 2024

(54) COMPOSITIONS AND METHODS USING A COMBINATION OF OLEUROPEIN AND QUERCETIN FOR CELLULAR ENERGY

(71) Applicant: SOCIETE DES PRODUITS NESTLE S.A., Vevey (CH)

(72) Inventors: UMBERTO DE MARCHI, Corminboeuf (CH); JEROME FEIGE, Crissier (CH)

(21) Appl. No.: 18/253,419

(22) PCT Filed: Nov. 16, 2021
(86) PCT No.: PCT/EP2021/081829

§ 371 Date: May 18, 2023

(30) Foreign Application Priority Data
Nov. 18, 2020 (EP) 20208389.5

(51) Int. Cl. A61K 31/352; A61K 31/7048; A61P 43/00

(52) U.S. Cl. CPC A61K 31/352; A61K 31/7048; A61P 43/00

(57) ABSTRACT

Composition comprising a combination of oleuropein or metabolite thereof and quercetin or derivative thereof are provided. The composition may be an oral nutritional composition, for example a nutritional supplement, an oral nutritional supplement, a food product, a food for special medical purpose (FSMP). The composition can be administered to an individual in need thereof for improving a physiological state linked to metabolic fatigue in one or more cells, (ii) increasing mitochondrial energy and mitochondrial calcium uptake in one or more cells, and (iii) increasing antioxidant capacity, reducing oxidative stress and/or enhancing mitochondrial function, (iv) treating or preventing a calcium deficiency/depletion disorder in an individual. Additionally, or alternatively, the method can treat or prevent a mitochondria-related disease or a condition associated with altered mitochondrial function in an individual in need thereof or at risk thereof.

### BACKGROUND

The present disclosure generally relates to compositions and methods that use a combination of oleuropein or metabolite thereof and quercetin or metabolite to manage energy at a cellular level. The compositions and methods can boost mitochondrial function and increase bioenergetics through activation of the mitochondrial calcium uniporter to thereby promote cellular activation, in some embodiments in an older adult or an elderly individual.

Population aging has been a remarkable demographic event. As the growth of the older population has outpaced the total population due to increased longevity, the proportion of older persons relative to the rest of the population has increased considerably due to decreased fertility rates. For example, one in every twelve individuals was at least 60 years of age in 1950, and one in every ten was aged 60 years or older by the end of 2000. By the end of 2050, the number of persons worldwide that is 60 years or over is projected to be one in every five.

Aged or aging individuals frequently suffer some degree of physical decline and/or cognitive impairment, including decline in cognitive function, that progresses with age, and age-related changes in brain morphology and cerebrovascular function are commonly observed. Cognitive decline has been consistently reported with aging across a range of cognitive domains including processing speed, attention, episodic memory, spatial ability and executive function. Brain imaging studies have revealed that these normal age-related cognitive declines are associated with decreases in both grey and white matter volume in the brain, with the fronto-striatal system most heavily compromised with aging. These decreases in cortical volume can be attributed to a number of detrimental cellular processes involved with normal aging, such as accumulation of damage by free radicals over time leading to oxidative damage, chronic low-grade inflammation, homocysteine accumulation (which when elevated are a risk factor for cognitive impairment and dementia), and decreased mitochondrial efficiency. In addition to direct cellular damage, the brain is also indirectly impaired by insults to micro-vascular structures. It is evident that the pathology of aging and also dementia involves a complexity of these interacting factors which are linked together. For example, mitochondrial dysfunction leads to increased oxidative stress, and oxidative stress can trigger inflammation and vascular insults.

Mitochondria are the primary source of aerobic energy production in mammalian cells and also maintain a large Ca2+ gradient across their inner membrane, providing a signaling potential for this molecule. Furthermore, mitochondrial Ca2+ plays a role in the mitochondria in the regulation of ATP generation and potentially contributes to the orchestration of cellular metabolic homeostasis. (Glancy, B. et al. (2012). “Role of mitochondrial Ca2+ in the regulation of cellular energetics.” Biochemistry 51(14): 2959-2973). Alterations in mitochondrial Ca2+ homeostasis have been linked to a variety of pathological conditions and are critical in the aetiology of several human diseases (Arduino et al. Journal Physiol. 2018 July; 596(14):2717-2733).

Nutrition, education, physical exercise and cognitive exercise have been recently demonstrated as possible intervention to prevent physical and cognitive decline with aging. An abundance of clinical, epidemiological, and individual evidence is in favor of individual nutritional factors that reduce dementia risk and age-related neurodegeneration. However, formal trial testing of nutritional interventions has yielded mixed results (Schmitt et al., Nutrition Reviews 68: S2-S5 (2010). Also, efforts have been taken to harness mitochondrial Ca2+ transport mechanisms for therapeutic intervention, but pharmacological compounds that direct and selectively modulate mitochondrial Ca2+ homeostasis are currently lacking.

### SUMMARY

In view of the experimental data disclosed later herein, the present inventors believe that a combination of oleuropein and quercetin enhances the efficiency of mitochondria to produce energy.

Accordingly, in a general embodiment, the present disclosure provides a composition comprising a combination of oleuropein and/or metabolite and quercetin and/or derivative in a therapeutically effective amount for use in improving a physiological state linked to metabolic fatigue in one or more cells, (ii) increasing mitochondrial energy and mitochondrial calcium uptake in one or more cells, and (iii) increasing antioxidant capacity, reducing oxidative stress and/or enhancing mitochondrial function, (iv) treating or preventing a calcium deficiency/depletion disorder in an individual.

In another embodiment, the present disclosure provides a composition comprising a combination of oleuropein and/or a metabolite thereof and quercetin and/or a derivative in a therapeutically effective amount for delaying off-set of metabolic decline, maintaining muscle mass and/or muscle function, decreasing oxidative stress, maintaining immune function and/or maintaining cognitive function in a healthy older adult.

In a further embodiment, the present disclosure also provides a composition comprising a combination of oleuropein and/or a metabolite thereof and quercetin and/or a derivative in a therapeutically effective amount for
i) enhancing at least one of mental performance or muscle performance in an individual or
ii) improving or maintaining cognitive function, in an individual.

In another embodiment, the invention provides a unit dosage form comprising a combination of a combination of oleuropein and/or a metabolite thereof and quercetin and/or a derivative in an amount effective for at least one of i) treating, reducing an incidence of, or reducing a severity of a mitochondria-related disease or condition associated with altered mitochondrial function (ii) improving in a physiological state linked to metabolic fatigue in one or more cells, (iii) increasing mitochondrial energy and mitochondrial calcium uptake in one or more cells, and (iv) treating or preventing a calcium deficiency/depletion disorder. (v) increasing metabolic rate, (vi) improving or maintaining cognitive function, (vii) increasing or maintaining mitochondrial function.

In another embodiment, the invention provides a kit comprising a combination of a combination of oleuropein and/or a metabolite thereof and quercetin and/or a derivative in one or more containers.

Advantages and additional features will be apparent from the following Figures and Detailed Description.

### BRIEF DESCRIPTION OF DRAWINGS

FIG. 1 is a graph showing that oleuropein (Ole) synergies with Quercetin (Q) to activate mitochondria via mitochondrial Ca2+ rise, during HeLa cells stimulation. The inset shows the effect of oleuropein (3 μM, black), quercetin (3 μM, gray) and the combination of 3 μM oleuropein+3 μM quercetin (Ole/Q) on the integrated mitochondrial calcium rise, evoked by 100 μM histamine. The data in the inset were used to determine, in the main figure, the expected theoretical effect (sum between oleuropein effect and quercetin effect) and the real measured effect of the combination (oleuropein+quercetin, Ole/Q) and to extrapolate the synergism. Results are expressed as mean+/−SEM from n=6-9 experiments. * indicates statistically significant difference of the measured vs. theoretical difference in mitochondrial calcium at P<0.05 (Student's t-test).

FIG. 2 is a graph showing that oleuropein aglycone (Oea) synergies with Quercetin (Q) to activate mitochondria via mitochondrial Ca2+ rise, in stimulated HeLa cells. The inset shows the effect of oleuropein aglycone (3 μM, black), quercetin (3 μM, gray) and the combination of 3 μM Oea+3 μM Q (Oea/Q) on the integrated mitochondrial calcium rise, evoked by 100 μM histamine. The data in the inset were used to determine, in the main figure, the expected theoretical effect (sum between Oea effect and quercetin effect) and the real measured effect of the combination (Oea+quercetin) and to extrapolate the synergism. Results are expressed as mean+/−SEM from n=6-9 experiments. * indicates statistically significant difference of the measured vs. theoretical difference in mitochondrial calcium at P<0.05 (Student's t-test).

FIG. 3 is a graph showing that Oleuropein (Ole) does not synergies with Oleuropein aglycone (Oea) to activate mitochondria via mitochondrial Ca2+ rise, in stimulated HeLa cells. The bar chart shows the effect of oleuropein (3 μM, black), oleuropein aglycone (3 μM, gray), and the combination of 3 μM Ole+3 μM Oea (Ole/Oea) on the integrated mitochondrial calcium rise, evoked by 100 μM histamine. Results are expressed as mean+/−SEM from n=6-9 experiments. * indicates statistically significant difference of the measured vs. theoretical difference in mitochondrial calcium at P<0.05 (one-way ANOVA test).

FIG. 4 is a graph showing that Oleuropein (Ole) and Oleuropein aglycone (Oea) promote the same level of synergism in combination with Quercetin (Q). The synergisms were calculated as described in FIG. 1 and FIG. 2. Results are expressed as mean+/−SEM from n=6 experiments. NS, not significant, indicates no statistically significant difference between the two group (combinations) at P<0.05 (Student's t-test).

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a metabolite” or “the metabolite” includes one metabolite but also two or more metabolites.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

As used herein, a “composition consisting essentially of at least one of oleuropein or metabolite thereof” and a “composition consisting essentially of calcium and at least one of oleuropein or metabolite thereof” do not include any additional compound that affects mitochondrial calcium import other than the at least one of oleuropein or metabolite thereof and the optional calcium. In a particular non-limiting embodiment, the composition consists of an excipient, the at least one of oleuropein or metabolite thereof, and optionally calcium.

The term “and/or” used in the context of “X and/or Y” should be interpreted as “X,” or “Y,” or “X and Y.” Similarly, “at least one of X or Y” should be interpreted as “X,” or “Y,” or “both X and Y.” For example, “at least one of oleuropein or metabolite thereof” means “oleuropein,” or “a metabolite of oleuropein,” or “both oleuropein and a metabolite thereof.”

Where used herein, the terms “example” and “such as,” particularly when followed by a listing of terms, are merely exemplary and illustrative and should not be deemed to be exclusive or comprehensive. As used herein, “associated with” and “linked with” mean occurring concurrently, preferably means caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development or progression of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

Both human and veterinary treatments are within the scope of the present disclosure. Preferably the at least one of oleuropein or metabolite thereof is administered in a serving or unit dosage form that provides a therapeutically effective or prophylactically effective amount.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

The relative terms “improved,” “increased,” “enhanced” and the like refer to the effects of the composition disclosed herein, namely a composition comprising an effective amount of at least one of oleuropein or metabolite thereof, relative to administration over the same time period of a composition lacking oleuropein and lacking an oleuropein metabolite but otherwise identical.

As used herein, “administering” includes another individual providing a referenced composition to an individual so that the individual can consume the composition and also includes merely the act of the individual themselves consuming a referenced composition.

“Animal” includes, but is not limited to, mammals, which includes but is not limited to rodents; aquatic mammals; domestic animals such as dogs, cats and other pets; farm animals such as sheep, pigs, cows and horses; and humans. Where “animal,” “mammal” or a plural thereof is used, these terms also apply to any animal that is capable of the effect exhibited or intended to be exhibited by the context of the passage, e.g., an animal benefitting from improved mitochondrial calcium import. While the term “individual” or “subject” is often used herein to refer to a human, the present disclosure is not so limited. Accordingly, the term “individual” or “subject” refers to any animal, mammal or human that can benefit from the methods and compositions disclosed herein.

The term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal. The term “companion animal” means a dog or a cat.

A “subject” or “individual” is a mammal, preferably a human. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly individuals. The term “older adult” in the context of a human means an age from birth of at least 45 years, preferably above 50 years, more preferably above 55 years, and includes elderly individuals.

As used herein, “frailty” is defined as a clinically recognizable state of increased vulnerability resulting from aging-associated decline in reserve and function across multiple physiologic systems such that the ability to cope with everyday or acute stressors is compromised. A pre-frail stage, in which one or two of these criteria are present, identifies a high risk of progressing to frailty.

The terms “serving” or “unit dosage form,” as used herein, are interchangeable and refer to physically discrete units suitable as unitary dosages for human and animal subjects, each unit containing a predetermined quantity of the composition comprising at least one of oleuropein or metabolite thereof, as disclosed herein, in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of liquid housed within a container such as a bottle.

An “oral nutrition supplement” or “ONS” is a composition comprising at least one macronutrient and/or at least one micronutrient, for example in a form of sterile liquids, semi-solids or powders, and intended to supplement other nutritional intake such as that from food. Non-limiting examples of commercially available ONS products include MERITENE®, BOOST®, NUTREN® and SUSTAGEN®. In some embodiments, an ONS can be a beverage in liquid form that can be consumed without further addition of liquid, for example an amount of the liquid that is one serving of the composition.

As used herein, “incomplete nutrition” refers to preferably nutritional products that do not contain sufficient levels of macronutrients (protein, fats and carbohydrates) or micronutrients to be sufficient to be a sole source of nutrition for the animal to which the nutritional product is being administered. The term “complete nutrition” refers to a product which is capable of being the sole source of nutrition for the subject. An individual can receive 100% of their nutritional requirements from a complete nutrition composition.

A “kit” means that the components of the kit are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof.

“Metabolic fatigue” means reduced mitochondrial function in one or more cells (e.g., one or more of liver, kidney, brain, skeletal muscle) due to a shortage of substrates within the one or more cells and/or an accumulation of metabolites within the muscle fiber which interfere either with the release of calcium or with the ability of calcium to stimulate mitochondrial function. Physiological states linked to metabolic fatigue may comprise muscle fatigue or weakness, lack of energy, in particular physical energy, lack of vitality or weakness.

### EMBODIMENTS

The present disclosure provides a composition comprising a combination of oleuropein and/or a metabolite thereof and quercetin and/or a derivative, in a therapeutically effective amount for use in (i) improving a physiological state linked to metabolic fatigue in one or more cells, (ii) increasing mitochondrial energy and mitochondrial calcium uptake in one or more cells, and (iii) increasing antioxidant capacity, reducing oxidative stress and/or enhancing mitochondrial function, (iv) treating or preventing a calcium deficiency/depletion disorder in an individual.

Oleuropein is a polyphenol found in the fruit, the roots, the trunk and more particularly in the leaves of plants belonging to the Oleaceae family, and especially Olea europaea.

In an embodiment, at least a portion of the oleuropein is obtained by extraction, e.g., by extraction from a plant such as a plant belonging to the Oleaceae family, preferably one or more of the stems, the leaves, the fruits or the stones of a plant belonging to the Oleaceae family such as Olea europaea (olive tree), a plant of genus Ligustrum, a plant of genus Syringa, a plant of genus Fraximus, a plant of genus Jasminum and a plant of genus Osmanthus. Additionally or alternatively, at least a portion of the oleuropein and/or metabolites can be obtained by chemical synthesis.

Non-limiting examples of suitable metabolites of oleuropein include oleuropein aglycone, hydroxytyrosol, elenolic acid, homovanillyl alcohol, isohomovanillyl alcohol, glucuronidated forms thereof, sulfated forms thereof, derivatives thereof, and mixtures thereof.

Quercetin is the aglycone form of a number of other flavonoid glycosides, such as rutin and quercitrin, found in citrus fruit, buckwheat and onions. Quercetin forms the glycosides quercitrin and rutin together with rhamnose and rutinose, respectively.

Likewise guaijaverin is the 3-O-arabinoside, hyperoside is the 3-O-galactoside, isoquercitin is the 3-O-glucoside and spiraeoside is the 4′-O-glucoside. Miquelianin is the quercetin 3-O-β-D-glucuronopyranoside.

In a preferred embodiment, the derivative of quercetin may be selected from the group consisting of quercetin 3-O-galactoside, quercetin 3-O-glucoside (izoquercetin), quercetin 3-O-xyloside, quercetin 3-O-rhamnoside (quercitrin), quercetin 3-O-glucuronide, quercetin 7-O-glucoside, quercetin 3-O-diglucoside, quercetin 3,4′-diglucoside, quercetin 3-O-rhamnoside-70-glucoside, quercetin 3-O-rutinoside (rutin), quercetin 3-O-6″-acetylglucoside, quercetin 3-methyl ether, quercetin 3,3′-dimethyl ether, and mixtures thereof.

The quercetin may be from any suitable source and may be isolated and/or chemically synthesized.

In a preferred embodiment oleuropein and quercetin and derivatives are obtained from plant sources. For example, oleuropein may be obtained from olive plants, rutin may be obtained from onions, quercetin may be obtained from onions, green tea, apples, berries, Ginkgo biloba, St. John's wort, American elder, buckwheat tea and others.

The composition may also comprise one or more additional bioactive compounds, such as one or more compounds selected from the group consisting of antioxidants, anti-inflammatory compounds, glycosaminoglycans, prebiotics, fibers, probiotics, fatty acids, enzymes, minerals, trace elements and vitamins. A “bioactive compound” is any compounds that contributes to the health of an individual or has an effect on the human body, beyond that of meeting basic nutritional need. The one or more additional bioactive compounds may be from a natural source. Thus, the compounds may be from extracts of plants, animals, fish, fungi, algae, or microbial fermentation. Minerals are considered to be from a natural source. In a preferred embodiment, enzymes may be proteases such as trypsin, or enzyme extracts such as bromelain, for example.

In another embodiment, the oleuropein and/or derivative can be provided by any of the compositions and methods disclosed by WO 2019/092068 and WO 2019/092066, each entitled “Bioconversion of oleuropein” and “Method of selecting a probiotic”, and WO 2019/092069 entitled “Homovanillyl alcohol (HVA), HVA isomer, methods of making compositions comprising such compounds, and methods of using such compounds”, each incorporated herein by reference in its entirety.

The effective amount of each of the oleuropein and/or metabolite thereof and quercetin and/or derivative thereof varies with the particular composition, the age and condition of the recipient, and the particular disorder or disease being treated. Nevertheless, in a general embodiment, 0.001 mg to 1.0 g can be administered to the individual per day, preferably from 0.01 mg to 0.9 g per day, more preferably from 0.1 mg to 750 mg per day, more preferably from 0.5 mg to 500 mg per day, and most preferably from 1.0 mg to 200 mg per day. Moreover, the inventors found that the active dose of oleuropein or derivative in the combination, may be lowered for an equal efficacy.

In some embodiments, the combination of oleuropein or metabolite and the quercetin or derivative is administered in a composition further comprising calcium. At least a portion of the calcium can be one or more calcium salts, such as calcium acetate, calcium carbonate, calcium chloride, calcium citrate, calcium glubionate, calcium gluconate, calcium lactate or mixtures thereof. In a general embodiment, 0.1 g to 1.0 g of the calcium is administered to the individual per day, preferably from 125 mg to 950 g of the calcium per day, more preferably from 150 mg to 900 mg of the calcium per day, more preferably from 175 mg to 850 mg of the calcium per day, and most preferably from 200 mg-800 mg of the calcium per day.

In an alternative embodiment, the combination of oleuropein and quercetin can be administered sequentially with calcium in separate compositions. The term “sequentially” means that the calcium and the at least one of oleuropein or metabolite thereof are administered in a successive manner such that the at least one of oleuropein or metabolite thereof is administered at a first time without the calcium, and the calcium is administered at a second time (before or subsequent to the first time) without the combination of oleuropein and quercetin. The time between sequential administrations may be, for example, one or several seconds, minutes or hours in the same day; one or several days or weeks in the same month; or one or several months in the same year.

In some embodiments, the oleuropein or metabolite thereof and the quercetin or derivative thereof are the only polyphenols in the composition and/or the only polyphenols administered to the individual.

The composition can comprise an effective amount of at least one of oleuropein or metabolite thereof. For example, a single serving or dose of the composition can comprise the effective amount, and a package can contain one or more of the servings or doses. Optionally the composition can further comprise calcium.

The composition can comprise a food additive selected from the group consisting of acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, vitamins, minerals and combinations thereof.

The combination of oleuropein or metabolite and the quercetin or derivative can be administered in any composition that is suitable for human and/or animal consumption. In a preferred embodiment, it is administered to the individual orally or enterally (e.g. tube feeding). For example, it can be administered to the individual in a beverage, a food product, a capsule, a tablet, a powder or a suspension.

Non-limiting examples of suitable compositions for the include food compositions, dietary supplements, dietary supplements (e.g., liquid ONS), complete nutritional compositions, beverages, pharmaceuticals, oral nutritional supplement, medical food, nutraceuticals, food for special medical purpose (FSMP), powdered nutritional products to be reconstituted in water or milk before consumption, food additives, medicaments, drinks, petfood, and combinations thereof.

Food products according to the present invention may include dairy products, such as fermented milk products, e.g., yoghurts, buttermilk, etc; ice creams; concentrated milk; milk; dairy creams; flavoured milk drinks; whey based drinks; toppings; coffee creamers; chocolate; cheese based products; soups; sauces; purees; dressings; puddings; custards; baby foods; nutritional formulas, such as those for complete nutrition, for example for infants, children, teenagers, adults, the elderly or the critically ill; cereals and cereal bars, for example.

Drinks may include for example milk- or yoghurt-based drinks, fermented milk, protein drinks, coffee, tea, energy drinks, soy drinks, fruit and/or vegetable drinks, fruit and/or vegetable juices.

The combination of oleuropein or metabolite and the quercetin or derivative can be administered in a food product further comprising a component selected from the group consisting of protein, carbohydrate, fat and mixtures thereof.

In an embodiment, the source of protein is preferably purified protein (i.e., isolated from the native food ingredient in which it was created). The protein content of the composition is preferably 20-99 wt. % of the composition, for example 20-90 wt. % of the composition, for example, 30-80 wt. % of the composition, for example 40-80 wt. % of the composition, for example 50-80 wt. %, for example 40-70 wt. % of the composition.

Non-limiting examples of suitable protein or sources thereof for use in the compositions include hydrolyzed, partially hydrolyzed or non-hydrolyzed proteins or protein sources. They may be derived from any known or otherwise suitable source such as milk (e.g., casein, whey), animal (e.g., meat, fish), cereal (e.g., rice, corn) or vegetable (e.g., soy, pea) sources. Combinations of sources or types of proteins may be used. Non-limiting examples of proteins or sources thereof include intact pea protein, intact pea protein isolates, intact pea protein concentrates, milk protein isolates, milk protein concentrates, casein protein isolates, casein protein concentrates, whey protein concentrates, whey protein isolates, sodium or calcium casemates, whole cow's milk, partially or completely defatted milk, yoghurt, soy protein isolates and soy protein concentrates, and combinations thereof. Combinations of sources or types of proteins may be used. Preferred proteins include pea protein, whey protein, soy protein and casein. Casein proteins may, for example, comprise sodium caseinate and calcium caseinate.

The source of protein may be provided by individual amino acids, polypeptides comprising amino acids, or mixtures thereof. For many muscle growth, muscle maintenance and/or muscle enhancement treatments, particular amino acids beneficial, for example L-arginine, L-glutamine, lysine and the branched-chain amino acids (i.e. leucine, isoleucine, and valine; in particular leucine and isoleucine). These particular amino acids may be provided as the source of protein or they may be additional to a main source of protein. Thus, the source of protein in the composition may include one or more branched-chain amino acids (leucine, isoleucine, and valine); one or both of L-arginine and L-glutamine; and lysine. In a preferred embodiment, the composition comprises whey protein and/or casein protein together with one or more individual amino acids, for example one or more of (or all of) leucine, isoleucine and L-arginine.

In an embodiment, the composition further comprises a medium-chain triglyceride, for example one or more of caproic acid, caprylic acid, capric acid and lauric acid. In an embodiment, the composition further comprises a phospholipid, for example phosphatidylcholine.

The composition may also contain a carbohydrate and/or a source of fat. Non-limiting examples of suitable fats include canola oil, corn oil and high-oleic acid sunflower oil. Non-limiting examples of suitable carbohydrates include sucrose, lactose, glucose, fructose, corn syrup solids, maltodextrins, and mixtures thereof. Additionally or alternatively, a dietary fiber may be added. Dietary fiber passes through the small intestine undigested by enzymes and functions as a natural bulking agent and laxative. Dietary fiber may be soluble or insoluble and generally ablend of the two types is preferred. Non-limiting examples of suitable dietary fibers include soy, pea, oat, pectin, guar gum, partially hydrolyzed guar gum, gum Arabic, fructo-oligosaccharides, acidic oligosaccharides, galacto-oligosaccharides, sialyl-lactose and oligosaccharides derived from animal milks. A preferred fiber blend is a mixture of inulin with shorter chain fructo-oligosaccharides. In an embodiment, the fiber content is between 2 and 40 g/L of the composition, for example between 4 and 10 g/L.

One or more other minerals additional to any calcium can be used in the composition. Non-limiting examples of suitable minerals include boron, chromium, copper, iodine, iron, magnesium, manganese, molybdenum, nickel, phosphorus, potassium, selenium, silicon, tin, vanadium, zinc, and combinations thereof.

One or more other vitamins can be used in the composition. Non-limiting examples of suitable vitamins include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), Vitamin C, Vitamin D, Vitamin E, Vitamin K, folic acid and biotin), and combinations thereof. “Vitamin” includes such compounds obtained naturally from plant and animal foods or synthetically made, pro-vitamins, derivatives thereof, and analogs thereof.

One or more food grade emulsifiers may be incorporated into the composition, such as diacetyl tartaric acid esters of mono- and di-glycerides, lecithin, and/or mono- and di-glycerides. Suitable salts and stabilizers may be included.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

### Method of Treatment

The compositions disclosed herein can be effective in (i) improving a physiological state linked to metabolic fatigue in one or more cells, (ii) increasing mitochondrial energy and mitochondrial calcium uptake in one or more cells, and (iii) increasing antioxidant capacity, reducing oxidative stress and/or enhancing mitochondrial function, (iv) treating or preventing a calcium deficiency/depletion disorder in an individual.

In an embodiment, at least a portion of the one or more cells are part of at least one body part selected from the group consisting of liver, kidney, brain and skeletal muscle.

In another embodiment, metabolic fatigue comprises lack of energy, in particular physical energy, lack of vitality or weakness.

In some embodiments, the methods comprise identifying the individual as having the condition or being at risk of the condition before the administration.

In another embodiment, the present disclosure provides a combination of oleuropein and/or a metabolite thereof and quercetin and/or a derivative for treating or preventing (e.g., reducing incidence and/or severity) a mitochondria-related disease or a condition associated with altered mitochondrial function in an individual in need thereof or at risk thereof. The method comprises orally administering an effective amount of at least one of oleuropein or metabolite thereof to the individual in need thereof or at risk thereof.

Without being bound by theory, it is believed that various types of stress result in stress injury to mitochondria, thereby reducing their ability to perform numerous functions essential to overall cell function. The methods disclosed herein can be useful for treating conditions involving stress injury to mitochondria, which injury may be manifest in any of a number of ways including, but not limited to, mitochondrial disease.

Mitochondrial diseases are the result of either inherited or spontaneous mutations in mitochondrial DNA or nuclear DNA which lead to altered functions of the proteins or RNA molecules that normally reside in mitochondria. Problems with mitochondrial function, however, may only affect certain tissues as a result of factors occurring during development and growth that are not yet fully understood. Even when tissue-specific isoforms of mitochondrial proteins are considered, it is difficult to explain the variable patterns of affected organ systems in the mitochondrial disease syndromes seen clinically.

Mitochondrial diseases result from failures of the mitochondria, specialized compartments present in every cell of the body except red blood cells. Mitochondria are responsible for creating more than 90% of the energy needed by the body to sustain life and support growth. When they fail, less and less energy is generated within the cell. Cell injury and even cell death follow. If this process is repeated throughout the body, whole systems begin to fail, and the life of the person in whom this is happening is severely compromised. Mitochondrial diseases primarily affect children, but adult onset is becoming more recognized.

Diseases of the mitochondria appear to cause the most damage to cells of the brain, heart, liver, skeletal muscles, kidney, and the endocrine and respiratory systems.

Many symptoms in mitochondrial disorders are non-specific. The symptoms may also show an episodic course, with periodic exacerbations. The episodic condition of migraine, as well as myalgia, gastrointestinal symptoms, tinnitus, depression, chronic fatigue, and diabetes, have been mentioned among the various manifestations of mitochondrial disorders in review papers on mitochondrial medicine. In patients with mitochondrial disorders, clinical symptomatology typically occurs at times of higher energy demand associated with physiological stressors, such as illness, fasting, over-exercise, and environmental temperature extremes. Furthermore, psychological stressors also frequently trigger symptomatology, presumably due to higher brain energy demands for which the patient is unable to match with sufficient ATP production.

Depending on which cells are affected, symptoms may include loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

Mitochondrial diseases include, without limitation, Alper's disease; Barth syndrome; beta-oxidation defects; carnitine deficiency; carnitine-acyl-carnitine deficiency; chronic progressive external ophthalmoplegia syndrome; co-enzyme Q10 deficiency; Complex I deficiency; Complex II deficiency; Complex III deficiency; Complex IV deficiency; Complex V deficiency; CPT I deficiency; CPT II deficiency; creatine deficiency syndrome; cytochrome c oxidase deficiency; glutaric aciduria type II; Keams-Sayre syndrome; lactic acidosis; LCHAD (long-chain acyl-CoA dehydrogenase deficiency); Leber's hereditary optic neuropathy; Leigh disease; lethal infantile cardiomyopathy; Luft disease; MAD (medium-chain acyl-CoA dehydrogenase deficiency); mitochondrial cytopathy; mitochondrial DNA depletion; mitochondrial encephalomyopathy, lactic acidosis, and stroke-like symptoms; mitochondrial encephalopathy; mitochondrial myopathy; mitochondrial recessive ataxia syndrome; muscular dystrophies, myoclonic epilepsy and ragged-red fiber disease; myoneurogenic gastrointestinal encephalopathy; neuropathy, ataxia, retinitis pigmentosa, and ptosis; Pearson syndrome; POLG mutations; pyruvate carboxylase deficiency; pyruvate dehydrogenase deficiency; SCHAD (short-chain acyl-CoA dehydrogenase deficiency); and very long-chain acyl-CoA dehydrogenase deficiency.

Accordingly, an aspect of the present disclosure is a composition in a unit dosage form comprising a combination of oleuropein and/or a metabolite thereof and quercetin and/or a derivative in an amount effective for treatment or prevention of at least condition selected from the group consisting of stress (e.g., early-life stress and/or effects therefrom), obesity, reduced metabolic rate, metabolic syndrome, diabetes mellitus, hyperlipidemia, neurodegenerative disease, cognitive disorder, stress-induced or stress-related cognitive dysfunction, mood disorder (e.g., stress-induced or stress-related mood disorder), anxiety disorder (e.g., stress-induced or stress-related anxiety disorder) and age-related neuronal death or dysfunction (e.g., age-related neuronal death or dysfunction not attributable to a specific neurodegenerative disease), trauma, infection (e.g. in ICU) or cancer.

Another aspect of the present disclosure is a method of treating at least condition selected from the group consisting of stress, obesity, reduced metabolic rate, metabolic syndrome, diabetes mellitus, cardiovascular disease, hyperlipidemia, neurodegenerative disease, cognitive disorder, stress-induced or stress-related cognitive dysfunction, mood disorder (e.g., stress-induced or stress-related mood disorder), anxiety disorder (e.g., stress-induced or stress-related anxiety disorder) and age-related neuronal death or dysfunction (e.g., age-related neuronal death or dysfunction not attributable to a specific neurodegenerative disease), trauma, infection (e.g. in ICU) or cancer in an individual having the at least one condition.

In an embodiment of these methods, the hyperlipidemia that is treated or prevented comprises hypertriglyceridemia. In an embodiment of these methods, the hyperlipidemia that is treated or prevented comprises elevated free fatty acids. In an embodiment of these methods, the age-related neuronal death or dysfunction that is treated or prevented is by administration of the composition to an older adult, such as an elderly individual.

The stress that is treated or prevented can be early-life stress, i.e., stress experienced while under the age of five years from birth. Early-life stress has been reported to have a significant detrimental effect on cognitive performance, including psychological parameters such as increased rates of or susceptibility to depression, anxiety, and abnormal risk-taking behavior. Increased rates of attention-deficit/hyperactivity disorder (ADHD), post-traumatic stress disorder (PTSD), and major depression have been reported in individuals having experienced early-life stress.

Another aspect of the present disclosure is a method of delaying off-set of metabolic decline, maintaining muscle mass, decreasing oxidative stress, maintaining immune function and/or maintaining cognitive function in a healthy older adult.

The compositions disclosed herein can also be used in the treatment of any of a variety of additional diseases and conditions in which defective or diminished mitochondrial activity participates in the pathophysiology of the disease or condition, or in which increased mitochondrial function will yield a desired beneficial effect. Non-limiting examples of such conditions include male infertility associated with diminished sperm motility, macular degeneration and other age-related and inherited eye disorders, and hearing loss (e.g., age-related hearing loss).

Yet another aspect of the present disclosure is a unit dosage form comprising a combination of oleuropein and/or a metabolite thereof and quercetin and/or a derivative in an amount effective for at least one of i) treating, reducing an incidence of, or reducing a severity of a mitochondria-related disease or condition associated with altered mitochondrial function (ii) improving in a physiological state linked to metabolic fatigue in one or more cells, (iii) increasing mitochondrial energy and mitochondrial calcium uptake in one or more cells, and (iv) treating or preventing a calcium deficiency/depletion disorder. (v) increasing metabolic rate, (vi) improving or maintaining cognitive function, (vii) increasing or maintaining mitochondrial function.

In an embodiment, the physiological state linked to metabolic fatigue comprises muscle fatigue or weakness, lack of energy, physical energy, lack of vitality or weakness.

In a further embodiment, the unit dosage form consists essentially of the combination of oleuropein and/or a metabolite thereof and quercetin and/or a derivative. In some embodiments, one or more of these compounds can be isolated compounds.

The present disclosure also provides a kit comprising a combination of a combination of oleuropein and/or a metabolite thereof and quercetin and/or a derivative in one or more containers. In an embodiment of the kit, the one or more containers comprise at least one first container that stores the oleuropein and/or metabolite separately from the quercetin and/or derivative, which is stored in at least one second container, and the kit further comprises instructions for admixing the oleuropein with the quercetin into a unit dosage form.

In an embodiment of the kit, the combination can be provided together in one or more prepackaged unit dosage forms, for example in separate containers that each contain a dried powder such that each container contains one prepackaged unit dosage form.

In another embodiment, the kit can comprise a plurality of compositions for admixing together to form one or more of the compositions disclosed herein. For example, the kit can contain two or more dried powders in separate containers relative to each other, the separate powders each containing a portion of the final unit dosage form. As anon-limiting example of such an embodiment, the kit can contain one or more first containers that house the oleuropein and can also contain one or more second containers that house the quercetin. The content of one of the first containers can be admixed with one of the second containers to form at least a portion of the unit dosage form of the composition.

The above examples of administration do not require continuous daily administration with no interruptions. Instead, there may be some short breaks in the administration, such as a break of two to four days during the period of administration. The ideal duration of the administration of the composition can be determined by those of skill in the art.

### EXAMPLES

The following non-limiting examples present experimental data supporting the compositions and methods disclosed herein.

### Example 1

To test the effect of oleuropein (or oleuropein aglycone), quercetin and their combination in living cells, the inventors measured mitochondrial calcium elevation in HeLa cells. HeLa cells were purchased from ATCC. HeLa cells were seeded in 96-well plates at a density of 50000 cells per well in minimal essential medium (DMEM, Gibco), high glucose, +10% fetal calf serum.

Mitochondrial calcium measurements were carried out using Hela cells infected with the adenovirus (from Sirion biotech) expressing the mitochondrially targeted calcium sensor mitochondrial mutated aequorin (Montero et al., 2004). For aequorin reconstitution, 24 hours after infection, cells were incubated for 2 h at room temperature (22±° C.) in standard medium (145 mM NaCl, 5 mM KCl, 1 mM MgCl₂, 1 mM CaCl₂, 10 mM glucose and 10 mM Hepes, pH 7.4) with 1 μM wild-type coelenterazine.

For treatment, compounds were directly added to the cell culture or myotubes cultures 2 hours before measurements. Luminescence was measured at the FLIPR Tetra Aequorin (Molecular Devices). Mitochondrial calcium rise was obtained by stimulating the cells with 100 M histamine. Calibration of the luminescence data into calcium concentration was carried out using an algorithm, as described previously (Alvarez & Montero, 2002). Custom module analysis based on Excel (Microsoft) and GhaphPad Prism 7.02 (GraphPad) software was used for quantification.

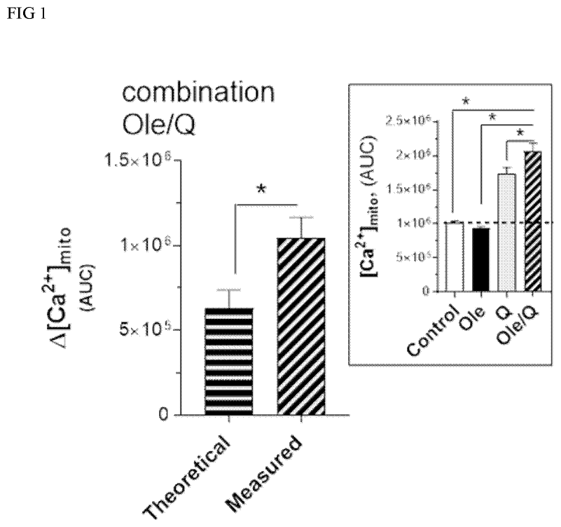

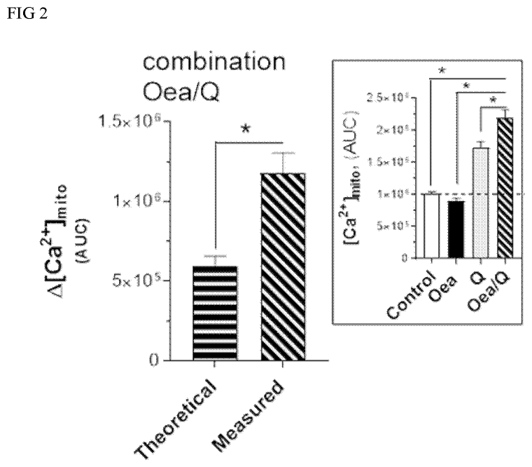

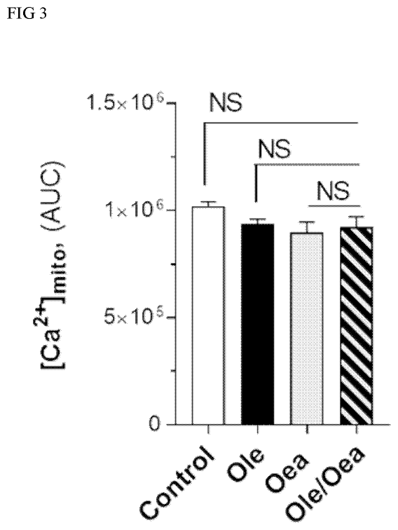

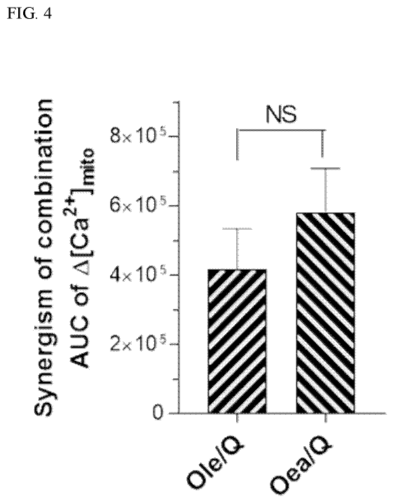

As shown in FIG. 1, oleuropein synergizes with quercetin to activate mitochondria, by increasing mitochondrial calcium elevation in Hela cells, during stimulation. As shown in FIG. 2, also oleuropein aglycone synergizes with quercetin to activate mitochondria, by increasing mitochondrial calcium elevation in Hela cells, during stimulation. Instead, as shown in FIG. 3, oleuropein does not synergize with oleuropein aglycone to activate mitochondria, via mitochondrial calcium elevation in Hela cells. As shown in FIG. 4, oleuropein and oleuropein aglycone promote the same level of synergism in combination with quercetin.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for (i) improving a physiological state linked to metabolic fatigue in one or more cells, (ii) increasing mitochondrial energy and mitochondrial calcium uptake in one or more cells, and (iii) increasing antioxidant capacity, reducing oxidative stress and/or enhancing mitochondrial function, (iv) treating or preventing a calcium deficiency/depletion disorder in an individual comprising administering to the individual a composition comprising a combination of oleuropein and/or a metabolite thereof and quercetin and/or a derivative, in a therapeutically effective amount.

2. The method according to claim 1, wherein at least a portion of the one or more cells are part of at least one body part selected from the group consisting of a liver, a kidney, a brain, and a skeletal muscle.

3. The method according to claim 1, wherein the physiological state linked to metabolic fatigue comprises muscle fatigue or weakness, lack of energy, physical energy, lack of vitality or weakness.

4. The method according to claim 1, wherein the effective amount of a combination of oleuropein and/or a metabolite thereof and quercetin and/or a derivative is orally administered daily for at least one week.

5. The method according to claim 1, wherein the metabolite of oleuropein is selected from the group consisting of oleuropein aglycone, hydroxytyrosol, elenolic acid, homovanillyl alcohol, isohomovanillyl alcohol, glucuronidated forms thereof, sulfated forms thereof, derivatives thereof, and mixtures thereof.

6. The method according to claim 1, wherein the derivative of quercetin is selected from the group consisting of quercetin 3-O-galactoside, quercetin 3-O-glucoside (izoquercetin), quercetin 3-O-xyloside, quercetin 3-O-rhamnoside (quercitrin), quercetin 3-O-glucuronide, quercetin 7-O-glucoside, quercetin 3-O-diglucoside, quercetin 3,4′-diglucoside, quercetin 3-O-rhamnoside-70-glucoside, quercetin 3-O-rutinoside (rutin), quercetin 3-O-6″-acetylglucoside, quercetin 3-methyl ether, quercetin 3,3′-dimethyl ether, and mixtures thereof.

7. The method according to claim 1, wherein the composition further comprises at least one compound selected from the group consisting of antioxidants, anti-inflammatory compounds, glycosaminoglycans, prebiotics, fibres, probiotics, fatty acids, enzymes, minerals, trace elements and/or vitamins.

8. The method according to claim 1, wherein the composition is selected from the group consisting of food compositions, dietary supplements, nutritional compositions, oral nutritional supplement, medical food, nutraceuticals, beverages, powdered nutritional products to be reconstituted in water or milk before consumption, food additives, food for special medical purpose (FSMP) medicaments, drinks, petfood, and combinations thereof.

9. The method according to claim 1, wherein the composition is in a form of a solid powder, a powdered stick, a capsule or a solution.

10. The use method according to claim 1, wherein the effective amount of a combination of oleuropein and/or a metabolite thereof and quercetin and/or a derivative is administered in a food product further comprising a component selected from the group consisting of protein, carbohydrate, fat and mixtures thereof.

11. A method for treating, reducing an incidence of, and/or reducing a severity of a mitochondria-related disease or a condition associated with altered mitochondrial function in an individual in need thereof or at risk thereof, the method comprising orally administering an effective amount of to the individual in need thereof or at risk thereof, the composition comprising a combination of oleuropein and/or a metabolite thereof and quercetin and/or a derivative in a therapeutically effective amount.

12. The method according to claim 11, wherein the mitochondria-related disease or condition is selected from the group consisting of stress, physiological ageing, obesity, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, musculoskeletal disorder, frailty, pre-frailty, chronic kidney disease, kidney failure, trauma, infection, cancer, hearing loss, macular degeneration, myopathies and dystrophies, and combinations thereof.

13. A method for delaying off-set of metabolic decline, maintaining muscle mass and/or muscle function, decreasing oxidative stress, maintaining immune function and/or maintaining cognitive function in a healthy older adult comprising administering a composition comprising a combination of oleuropein and/or a metabolite thereof and quercetin and/or a derivative in a therapeutically effective amount.

14-24. (canceled)
